(19) United States
(12) Patent Application Publication
(10) Pub. No.: US 2015/0160474 A1
(43) Pub. Date: Jun. 11, 2015

(54) CORRECTIVE LENS PRESCRIPTION ADAPTATION SYSTEM FOR PERSONALIZED OPTOMETRY

(71) Applicant: INTERNATIONAL BUSINESS MACHINES CORPORATION, ARMONK, NY (US)

(72) Inventors: Hung-Yang Chang, Scarsdale, NY (US); Lih Guong Jang, Hsinchu (TW); Joe Lu, Taipei (TW); Yi-Chang Wang, Hsinchu (TW); Nien-Chu Wu, Hsinchu (TW)

(73) Assignee: INTERNATIONAL BUSINESS MACHINES CORPORATION, ARMONK, NY (US)

(21) Appl. No.: 14/097,810

(22) Filed: Dec. 5, 2013

(51) Int. Cl. G02C 7/02; A61B 3/113; A61B 3/14; A61B 3/00

(52) U.S. Cl. CPC G02C 7/024; A61B 3/0008; A61B 3/113; A61B 3/14

(57) ABSTRACT

A method, system, and computer program product for adapting a corrective lens prescription. The system includes: an image capturing device; a lighting control device; a display; a memory; and a processor communicatively coupled to the image capturing device, the light generating device, the display, and the memory, wherein the processor is configured to perform the steps of a method including: generating a digital simulation for optometry, wherein the simulation is generated based on at least one lighting parameter, at least one activity parameter, at least one duration parameter, at least one personal parameter, and at least one target vision condition; recording user pupillary movement during the simulation; analyzing the recorded pupillary movement to determine a visual comfort assessment; and generating a message of the visual comfort assessment.

### BACKGROUND OF THE INVENTION

1. Field of the Invention

The present invention relates to ophthalmology/optometry and corrective lens fitting. More specifically, the present invention relates to automated corrective lens prescription adaptation.

2. Description of Related Art

After a corrective lens prescription is created, a trial lens is available for making adaptation adjustments. The optical setting of the trial lens is determined by the combined results of an auto-refractometer machine, an optometrist's diagnosis, and then the lens is given to the patient to test the comfort level. The common optometrist practice is to simply adjust the optometry prescription using the oral feedback of the comfort level self-assessment by the patient wearing the trial lens.

### SUMMARY OF THE INVENTION

According to a first aspect of the present invention, a method of adapting a corrective lens prescription is provided. The method includes: generating a digital simulation for optometry, wherein the simulation is generated based on at least one lighting parameter, at least one activity parameter, at least one duration parameter, at least one personal parameter, and at least one target vision condition; recording user pupillary movement during the simulation; analyzing the recorded pupillary movement to determine a visual comfort assessment; and generating a message of the visual comfort assessment.

According to another aspect of the present invention, a corrective lens prescription adaptation system is provided. The system includes: an image capturing device; a lighting control device; a display; a memory; and a processor communicatively coupled to the image capturing device, the lighting control device, the display, and the memory, wherein the processor is configured to perform the steps of a method including: generating a digital simulation for optometry, wherein the simulation is generated based on at least one lighting parameter, at least one activity parameter, at least one duration parameter, at least one personal parameter, and at least one target vision condition; recording user pupillary movement during the simulation; analyzing the recorded pupillary movement to determine a visual comfort assessment; and generating a message of the visual comfort assessment.

According to yet another aspect of the present invention, a computer program product for adapting a corrective lens prescription is provided. The computer program product includes: a computer readable storage medium having program code embodied therewith, the program code readable/executable by a processor to perform a method including: generating a digital simulation for optometry, wherein the simulation is generated based on at least one lighting parameter, at least one activity parameter, at least one duration parameter, at least one personal parameter, and at least one target vision condition; recording user pupillary movement during the simulation; analyzing the recorded pupillary movement to determine a visual comfort assessment; and generating a message of the visual comfort assessment.

### BRIEF DESCRIPTION OF THE DRAWINGS

FIG. 1 depicts a flowchart of a method of adapting a corrective lens prescription, according to an embodiment of the present invention.

FIG. 2 shows a detailed flowchart of a method of adapting a corrective lens prescription, according to an embodiment of the present invention.

FIG. 3 illustrates a detailed flowchart of the environmental-scenario simulation, according to an embodiment of the present invention.

FIG. 4 depicts a flowchart of the vision behavior modeling, according to an embodiment of the present invention.

FIG. 5 shows a flowchart of the visual comfort analysis, according to an embodiment of the present invention.

FIG. 6 shows a diagram of an exemplary computer system/server which is applicable to implements an embodiment of the present invention.

### DETAILED DESCRIPTION OF THE PREFERRED EMBODIMENTS

As will be appreciated by one skilled in the art, aspects of the present invention can be embodied as a system, method, or computer program product. Accordingly, aspects of the present invention can take the form of an entirely hardware embodiment, an entirely software embodiment (including firmware, resident software, micro-code, etc.) or an embodiment combining software and hardware aspects that can all generally be referred to herein as a “circuit,” “module” or “system.” Furthermore, aspects of the present invention can take the form of a computer program product embodied in one or more computer readable medium(s) having computer readable program code embodied thereon.

Any combination of one or more computer readable medium(s) can be utilized. The computer readable medium can be a computer readable signal medium or a computer readable storage medium. A computer readable storage medium can be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium can include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium can be any tangible medium that can contain, or store a program for use by or in connection with an instruction execution system, apparatus, or device.

A computer readable signal medium can include a propagated data signal with computer readable program code embodied therein, for example, in baseband or as part of a carrier wave. Such a propagated signal can take any of a variety of forms, including, but not limited to, electro-magnetic, optical, or any suitable combination thereof. A computer readable signal medium can be any computer readable medium that is not a computer readable storage medium and that can communicate, propagate, or transport a program for use by or in connection with an instruction execution system, apparatus, or device.

Program code embodied on a computer readable medium can be transmitted using any appropriate medium, including but not limited to wireless, wireline, optical fiber cable, RF, etc., or any suitable combination of the foregoing.

Computer program code for carrying out operations for aspects of the present invention can be written in any combination of one or more programming languages, including an object oriented programming language such as Java, Smalltalk, C++ or the like and conventional procedural programming languages, such as the “C” programming language or similar programming languages. The program code can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions can also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions can also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The present invention provides an automatic approach to reach an optimized comfort level of prescribed corrective lenses by using an interactive system that simulates target scenarios to test different levels of user comfort, and then analyzing patient response to the simulation.

The present invention predicts patient comfort level while they wear a prescription corrective lens. The patient views a digital animated simulation, which seeks to replicate experiences that the patient encounters in daily life. The simulation includes a dynamic design pattern. In traditional vision tests, a static design pattern is used to test visual comfort. The present invention utilizes dynamic patterns and animations to create a game-like environment that a patient interacts with. The system automatically monitors and measures the comfort level of the patient wearing a trial corrective lens while the individual interacts with the simulation.

Ophthalmologists/optometrists can adjust a patient's prescription based on the results of the generated visual comfort assessment message, rather than relying on verbal patient feedback.

According to an embodiment of the present invention, the system generates simulated three-dimensional (3D) images that correspond to daily life activities and lighting parameters, which become the content of an interactive simulation played by a patient wearing trial corrective lenses. The system records and analyzes the simulation playing behaviors of the patient, which an optometrist can reference. The system assesses the comfort level of the patient wearing the lenses and generates a message and a set of usage guidelines for the corrective lenses. The system includes an environmental-scenario simulation module and a prescription adaptation module. The environmental-scenario simulation module includes databases of template images and target vision conditions. It also includes a vision behavior modeler. Based on input data, the environmental-scenario simulation module generates images. The patient then interacts with the simulation and his/her behavior and pupil activity are recorded. Based on the behavior and activity, the patient's visual comfort is analyzed. From the results of the visual comfort analysis, the system generates a message indicating a positive comfort level assessment or a negative comfort level assessment. If a negative comfort level assessment message is generated, the corrective lens prescription is adjusted and the patient's pupillary activity and behavior are captured and analyzed while he/she views the simulation again.

In order to generate images and lighting parameters in the environmental-scenarios simulation, a set of data parameters are collected from the patient and input. Lifestyle parameters, which include the activities that the patient performs on a daily basis, are collected and input. For example, a patient can use corrective lenses for reading purposes only. Or a patient can use corrective lenses only while driving a motor vehicle. A patient can spend a majority of their time inside or outdoors. Based on these daily activities, lighting parameters and the size and movement of objects likely to be encountered daily can be determined. Also, the duration the patient spends performing the daily activities can be collected. The activities that an individual performs on a daily basis needs to be collected in order to generate simulations that the patient would most likely encounter while wearing corrective lenses. In order to collect these lifestyle parameters, the patients are interviewed about their daily activities. The collected parameters are input into the environmental-scenario simulation.

A further input into the environmental-scenario simulation can include individual facts about the patient and personal preference factors. The patient's age, previous prescription parameters, and any know eye diseases the patient has can be input into the environmental-scenario simulator. These personal parameters further aid in generating a simulated environment that closely resembles a patient's daily activities. An optometry prescription can include up to four parameters, pupillary distance (PD), spherical power (Sph), cylindrical power (Cyl), and axis of focus (Axis), all of which can be input as a patient's personal parameters.

Based on the daily activity and lighting inputs and the personal factor inputs, the environment-scenario simulation generates a corresponding parametric rule set to control the generation of the animated environment-scenario. To generate the images, the environment-scenario simulation accesses a database, which can contain stored images that an individual is likely to encounter on a daily basis. The database can provide images that relate to different activities and different lighting levels, and it can contain different objects that can be used in the simulation. The graphical templates in the database are accessed based on the parametric rule set developed from the information collected from the patient and input into the environment-scenario simulation. The templates are then used to generate the simulation. Also contained in the database are target vision conditions. These conditions are the target conditions that indicate visual comfort, and they represent the conditions that an eye experiences when it is comfortable. The target vision conditions compute rules that influence how objects behave. These conditions influence the lighting, speed, angular movement, and perceived distance of the objects that appear during the simulation. The environment-scenario simulation can also control the ambient light in the simulation. The ambient light can be adjusted to simulate daily lighting levels that a patient is likely to encounter.

The environment-scenario simulation can include a vision behavior modeler. The vision behavior modeler takes into account object size, object illumination, and object attributes. In order to generate the simulation of the daily life activities, these three elements are used to compute rule sets that govern the relationship of objects and their visual characteristics. The objects' size, illumination, and attributes are determined from the patient's lifestyle parameters. The spatial-temporal rendering of an object is determined by distance and angle rules. The distance rule determines how near or far an object appears. The size of the object appearing in the simulation can be used to simulate how near or far an object appears to the patient. The angle rule determines how high or low an object appears. For example, if an individual uses corrective lenses while reading, he/she experiences images at low angles and near distances. An individual who uses corrective lenses while driving, experiences images at high angles and far distances. The vision behavior modeler takes into account the lifestyle parameters of the patient, and the activities they perform while wearing corrective lenses. The vision behavior modeler also takes into account the illumination of objects that an individual is likely to encounter in their daily activities. The vision behavior modeler can be used to simulate special interrupt events or an effect rule. The modeler can then govern how an object appears and behaves during emergency conditions. This can be used to simulate events that an individual can encounter in daily life. After the visual characteristics are computed by the vision behavior modeler, the simulations of the daily activities are generated according to the computations.

The images of the simulation are generated on a display and viewed by the patient. The images include objects that move according to the computations of the vision behavior modeler. While the patient wears a trial corrective lens, the objects generated move and change sizes. While the patient follows the objects with their eyes, an image capturing device, such as a camera, records the activity of the patient's pupil. The recording device captures and records the pupil's movement, which is then used to determine the comfort level of the patient. Instead of passively viewing the simulation, the patient can also interactive with the simulated environment. The patient can control an input device and can respond to the images by using the input device. The patient's interactions with the simulation can be captured and then analyzed as part of a visual comfort analysis.

The visual comfort analysis analyzes the captured pupillary movements of the patient while he/she views the simulation. The analysis takes into consideration the target vision conditions. The visual comfort analysis measures certain changes that take place in the eye as the objects and images of the simulation move and change. When a simulated object moves and its perceived distance changes, the eye changes the form of the elastic lens in order to maintain focus on the image. These changes can be measured by analyzing the size of the pupil opening and the swing of the pupil as it tracks moving objects at different perceived distances.

The iris controls the pupil opening and thus the amount of light entering the retina. By measuring the size of the iris as the objects of the simulation move, the amount of light that is allowed to enter the eye can be determined. Thus, the size of the iris and the light that is accommodated to enter the eye can be cross-correlated and the result used as an indication of visual comfort. When the eye is focused on a moving object at certain distances, the eye shows a pupil swing frequency response in an attempt to maintain object clarity. By measuring the movement of objects at different perceived distances and tracking how the eye follows the objects, the clarity of the object viewed by the patient can be determined. Thus, the movement of the objects during the simulation and the perceived distance can be cross-correlated and the result used as an indication of visual comfort. The visual comfort analysis uses these measurements, along with the target vision conditions to compute the comfort level of the patient while he/she views the simulation. Based on the analysis, the system determines whether the pupillary activity reflects a certain visual comfort level. If the pupillary movements are not stable or fail to meet the target vision conditions, the system generates a message indicating a negative comfort level. Based on this message, the optometrist or ophthalmologist can adjust the corrective lens prescription or provide a new corrective lens and have the patient interact with the system again. If the pupillary movements are stable or meet the target vision conditions, the system generates a message indicting a positive comfort level and a set of usage guidelines to be followed by the patient.

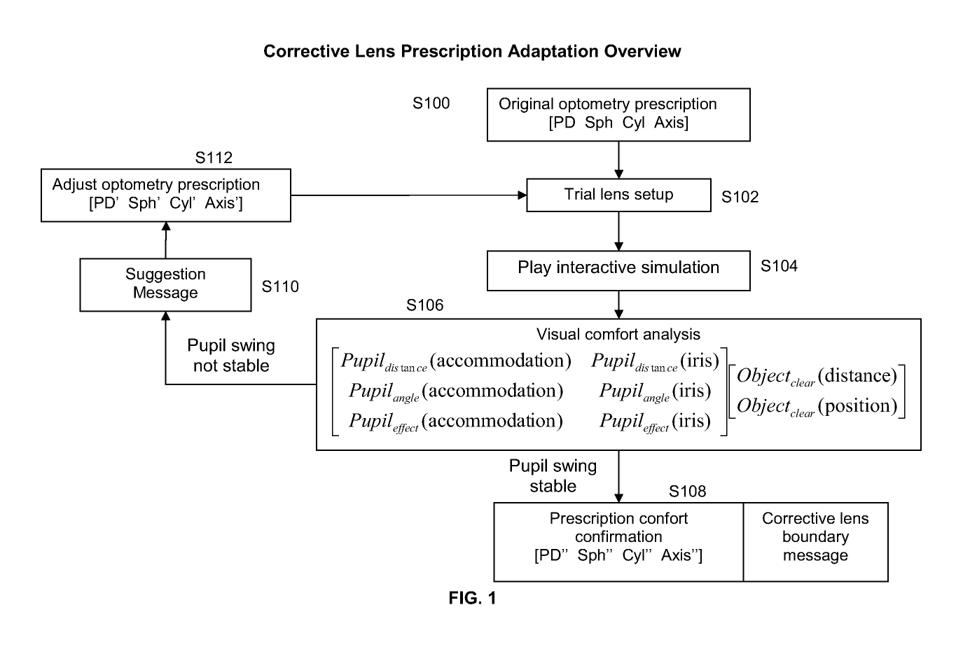

According to an embodiment of the present invention, FIG. 1 shows an overview of a method of adapting a corrective lens prescription. In S100, parameters of a patient's optometry prescription are determined and a trial corrective lens is fabricated in S102. While wearing the trial corrective lens, the patient views an interactive simulation in S104 that is generated by the environments-scenario simulation module. The patient can interact with the simulation by controlling an input device, such as a mouse, joystick, or other control device. While the patient interacts and views the simulation, a camera, sensor, or other recording device records the behaviors of the eye. In S106, a visual comfort analysis then analyzes the pupillary movements of the eye while the patient interacts with the animated simulation. During the visual comfort analysis, pupil response and object clarity are measured by cross-correlating light accommodation with iris size and object distance with object position. The pupil response measurements are analyzed based on the rules of distance, angle, and effect that govern vision behavior modeling.

If the pupil movements are stable throughout the course of the simulation, a message indicates that the trial lens prescription is accurate and provides the patient with a visual comfort confirmation message in S108. If the pupil movements are not stable, a message is displayed that suggests potential modifications to the prescription in S110. The optometry prescription can then be adjusted in S112 and a new trial lens can be fabricated in S102. The patient then views and interacts with the simulation and the process repeats until a prescription comfort confirmation message is generated.

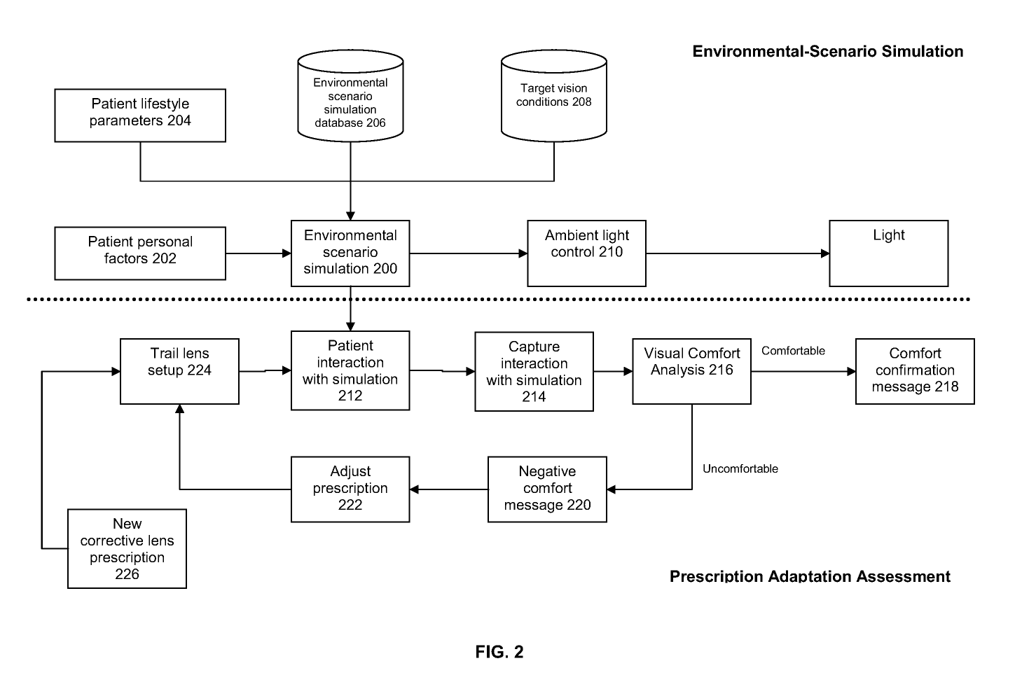

According to an embodiment of the present invention, FIG. 2 shows a detailed overview of a method of adapting a corrective lens prescription. The portion of the flowchart above the dotted line further illustrates the environmental-scenario simulation and the portion below the dotted line further illustrates prescription adaptation assessment. To generate environment-scenario simulation 200, patient personal factors 202 and patient lifestyle parameters 204 are collected. Environment-scenario simulation 200 receives images and objects in environmental scenario simulation database 206, as well as, target vision conditions 208. Based on these parameters, environmental scenario simulation 200 computes a rule set that controls the generation of a digital animated simulation. Environmental-scenario simulation 200 generates images that correspond to different activities, and it generates lighting conditions by controlling ambient light control 210 to simulate lighting conditions encountered by the patient.

While environmental-scenario simulation 200 is generated, a patient interacts with the simulation in 212. The patient interacts by viewing the digital simulation or by using a device that allows him/her to actively participate in the simulation. A device can be used to capture or record the patient's interaction with the simulation in 214. The captured activity can include pupillary movement and/or the device the patient is using to interact with the simulation. Based on the data collected from the interaction with the simulation in 214, a visual comfort analysis determines the level of comfort of the patient in 216. If the visual comfort analysis determines that the patient is comfortable while viewing the simulation, a comfort confirmation message is generated in 218. If the visual comfort analysis determines that the patient is uncomfortable, a negative comfort message is generated in 220. If a negative comfort message is generated, an optometrist or ophthalmologist can adjust the corrective lens prescription worn by the patient in 222. With the adjusted prescription, a trial lens is setup in 224 and the patient interacts and views the simulation again in 212.

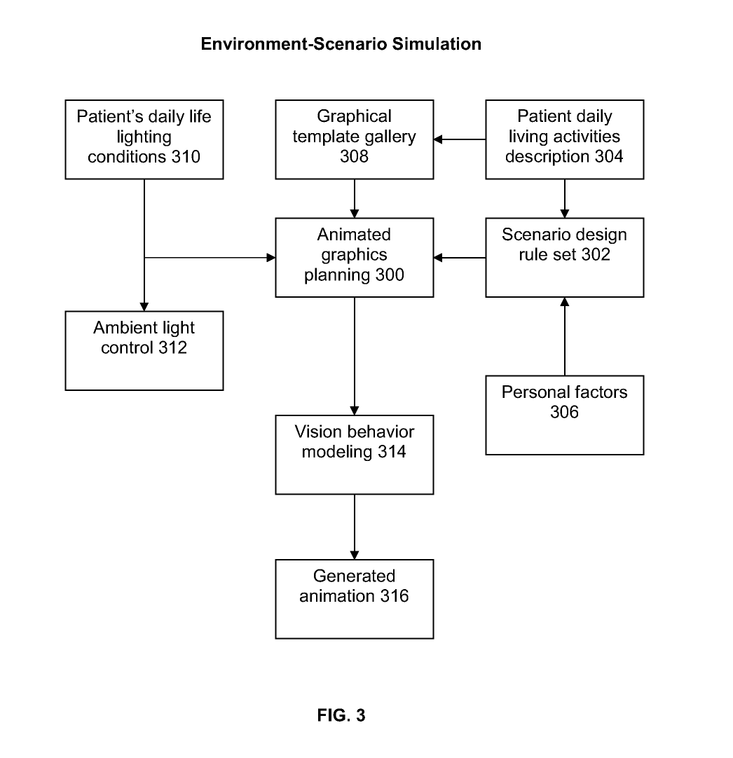

According to an embodiment of the present invention, FIG. 3 depicts a flowchart of generating the environment-scenario simulation. Animated graphics planning receives data and images from different inputs in 300. Animated graphics planning receives a scenario design rule set which is computed in 302. The scenario design rule set is computed from patient daily living activities input in 304, and from a patient's personal factors input in 306. Animated graphics planning receives images from a graphical template gallery in 308, which includes animated objects and background designs based on the patient's daily activities description input in 304. Animated graphic planning receives the patient's daily lighting conditions input in 310. These daily lighting conditions allow the environment scenario simulation to control the ambient light in 312. With these inputs, a relationship between the display background and animated objects is determined in 300. Next in 314, a visual behavior modeler is used to compute the spatial-temporal position, duration, and movement of the animated objects during the simulation. Finally, the environment-scenario simulation generates the digital animation in 316.

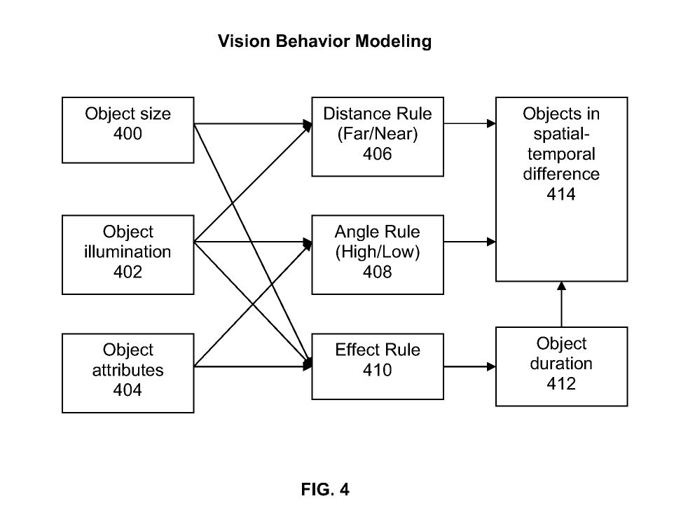

According to an embodiment of the present invention, FIG. 4 depicts a flowchart of the vision behavior modeling. Vision behavior modeling utilizes three elements: object size 400, object illumination 402, and object attributes 404. These elements are used to compute rule sets that govern the relationship of objects and their visual characteristics. The rule sets are used to generate objects during the simulation at certain spatial-temporal positions, for certain durations, and that certain angular movements. Object size 400 and object illumination 402 are used to compute distance rule 406. By using objects of certain sizes and certain lighting levels, the vision behavior modeling can determine how near or far an object appears to the patient viewing the simulation. Object illumination 402 and object attributes 404 are used to compute angle rule 408. By using objects with certain lighting levels and certain visual qualities, the vision behavior modeling can determine how high or low an object appears during the simulation. Object size 400, object illumination 402, and object attributes 404 are used to compute effect rule 410. Effect rule 410 determines how an object behaves during emergency conditions, such as a sudden lighting interference or sound event. These special interrupting events can appear for a short duration, thus effect rule 410 can be used to determine the duration an object will appear in the simulation. With all these rules computed, object spatial-temporal differences 414 are computed based on distance rule 406 and angle rule 408. The duration the object appears in the simulation is computed based on object duration 412. Thus, vision behavior modeling controls the movement and position of objects generated during the environment-scenario simulation.

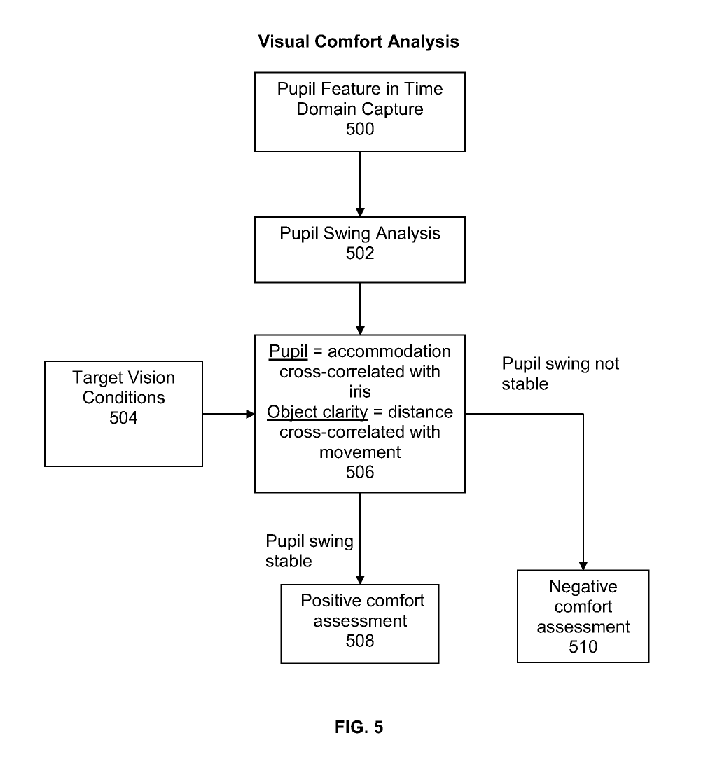

According to an embodiment of the present invention, FIG. 5 shows a flowchart of visual comfort analysis. The visual comfort analysis analyzes the pupillary movements of the patient while viewing the interactive simulation. In 500, an image capturing device will record the pupil's features during the simulation. During the simulation, the pupil movements and how clear an object appears are measured in 502. The measurements include the amount of light that is accommodated to enter the iris, by measuring the size of the iris, and the pupil swing frequency response, by tracking the trajectory of objects in the simulation and the pupil movement. With the measurements obtained in 502 and the target vision conditions received from a database in 504, the visual comfort of a patient is analyzed in 506. In 506, the amount of light accommodated to enter the eye is cross-correlated with the size of the iris and the distance of an object is cross-correlated with the movement of the object. From these correlations, the visual comfort analysis calculates whether the patient is experiencing visual comfort. If the pupil swing is stable and within the target vision conditions, a positive comfort assessment message is generated in 508. If the pupil swing is not stable and fails to meet the target vision conditions, a negative comfort assessment message is generated in 510. When a negative message is generated, the corrective lens prescription is adjusted and the patient views the interactive simulation until a positive comfort assessment message is obtained.

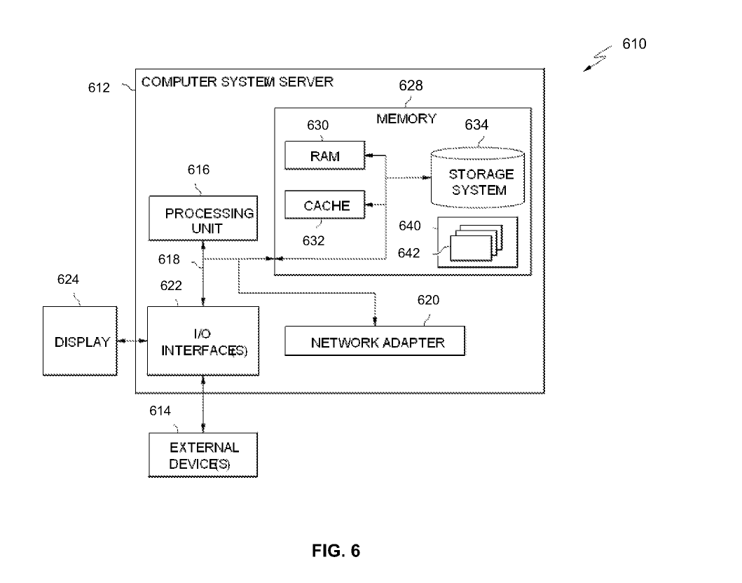

FIG. 6 shows a block diagram of an exemplary computer system/server 610 which is applicable to implement the embodiments of the present invention. The computer system/server 610 shown in FIG. 6 is only illustrative and is not intended to suggest any limitation as to the scope of use or functionality of embodiments of the invention described herein.

As shown in FIG. 6, computer system/server 612 is shown in the form of a general-purpose computing device. The components of computer system/server 612 can include, but are not limited to, one or more processors or processing units 616, a system memory 628, and a bus 18 that couples various system components including system memory 628 to processor 616.

Bus 618 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus. Computer system/server 612 typically includes a variety of computer system readable media. Such media can be any available media that is accessible by computer system/server 612, and it includes both volatile and non-volatile media, removable and non-removable media.

System memory 628 can include computer system readable media in the form of volatile memory, such as random access memory (RAM) 630 and/or cache memory 632. Computer system/server 612 can further include other removable/non-removable, volatile/non-volatile computer system storage media. By way of example only, storage system 634 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 628 can include at least one program product having a set (e.g., at least one) of program modules that are configured to carry out the functions of embodiments of the invention.

Program/utility 640, having a set (at least one) of program modules 642, can be stored in memory 628, by way of example, and not limitation, as well as an operating system, one or more application programs, other program modules, and program data. Each of the operating system, one or more application programs, other program modules, and program data or some combination thereof, can include an implementation of a networking environment. Program modules 642 generally carry out the functions and/or methodologies of embodiments of the invention as described herein.

Computer system/server 612 can also communicate with one or more external devices 614 such as a keyboard, a pointing device, an image capturing device, a lighting control device, a display 624, etc.; one or more devices that enable a user to interact with computer system/server 612; and/or any devices (e.g., network card, modem, etc.) that enable computer system/server 612 to communicate with one or more other computing devices. Such communication can occur via Input/Output (I/O) interfaces 622. Still yet, computer system/server 612 can communicate with one or more networks such as a local area network (LAN), a general wide area network (WAN), and/or a public network (e.g., the Internet) via network adapter 620. As depicted, network adapter 620 communicates with the other components of computer system/server 612 via bus 618. It should be understood that although not shown, other hardware and/or software components can be used in conjunction with computer system/server 612. Examples, include, but are not limited to: microcode, device drivers, redundant processing units, external disk drive arrays, RAID systems, tape drives, and data archival storage systems, etc.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of adapting a corrective lens prescription, the method comprising:
generating a digital simulation for optometry, wherein the simulation is generated based on at least one lighting parameter, at least one activity parameter, at least one duration parameter, at least one personal parameter, and at least one target vision condition;
recording user pupillary movement during the simulation;
analyzing the recorded pupillary movement to determine a visual comfort assessment; and
generating a message of the visual comfort assessment.

2. The method according to claim 1, wherein generating the digital simulation comprises:
receiving the at least one lighting parameter, the at least one activity parameter, the at least one duration parameter, the at least one personal parameter, and the at least one target vision condition;
generating a parameter rule set based on the parameters received to determine a relationship between a display background and a plurality of images, wherein the display background and the plurality of images are selected from a database;
computing a visual behavior function; and
generating the digital simulation.

3. The method according to claim 2, wherein computing the visual behavior function comprises:
computing a spatial-temporal position of an object, wherein the spatial-temporal position of the object includes at least one rule of distance and at least one rule of angle; and
computing a duration of the object, wherein the duration of the object includes at least one rule of position and at least one effect rule.

4. The method according to claim 1, wherein analyzing the recorded pupillary movement comprises:
measuring a size of the user pupillary opening;
measuring the user pupillary movement and perceived distance and movement of objects generated in the simulation; and
determining the visual comfort assessment, wherein the visual comfort assessment is either positive or negative.

5. The method according to claim 4, wherein the message of the visual comfort assessment comprises either a positive visual comfort assessment and a set of usage guidelines, or a negative visual comfort assessment.

6. The method according to claim 1, wherein the user wears a prescription corrective lens.

7. A corrective lens prescription adaptation system comprising:
an image capturing device;
a lighting control device;
a display;
a memory; and
a processor communicatively coupled to the image capturing device, the light generating device, the display, and the memory, wherein the processor is configured to perform the steps of a method comprising:
generating a digital simulation for optometry, wherein the simulation is generated based on at least one lighting parameter, at least one activity parameter, at least one duration parameter, at least one personal parameter, and at least one target vision condition;
recording user pupillary movement during the simulation;
analyzing the recorded pupillary movement to determine a visual comfort assessment; and
generating a message of the visual comfort assessment.

8. The system according to claim 7, wherein generating the digital simulation comprises:
receiving the at least one lighting parameter, the at least one activity parameter, the at least one duration parameter, the at least one personal parameter, and the at least one target vision condition;
generating a parameter rule set based on the parameters received to determine a relationship between a display background and a plurality of images, wherein the display background and the plurality of images are selected from a database;
computing a visual behavior function; and
generating the digital simulation.

9. The system according to claim 8, wherein computing the visual behavior function comprises:
computing a spatial-temporal position of an object, wherein the spatial-temporal position of the object includes at least one rule of distance and at least one rule of angle; and
computing a duration of the object, wherein the duration of the object includes at least one rule of position and at least one effect rule.

10. The system according to claim 7, wherein analyzing the recorded pupillary movement comprises:
measuring a size of the user pupillary opening;
measuring the user pupillary movement and perceived distance and movement of objects generated in the simulation; and
determining the visual comfort assessment, wherein the visual comfort assessment is either positive or negative.

11. The system according to claim 10, wherein the message of the visual comfort assessment comprises either a positive visual comfort assessment and a set of usage guidelines, or a negative visual comfort assessment.

12. The system according to claim 7, wherein the user wears a prescription corrective lens.

13. A computer program product for corrective lens prescription adaptation, the computer program product comprising a computer readable storage medium having program code embodied therewith, the program code readable/executable by a processor to perform a method comprising:
generating a digital simulation for optometry, wherein the simulation is generated based on at least one lighting parameter, at least one activity parameter, at least one duration parameter, at least one personal parameter, and at least one target vision condition;
recording user pupillary movement during the simulation;
analyzing the recorded pupillary movement to determine a visual comfort assessment; and
generating a message of the visual comfort assessment.

14. The computer program product according to claim 13, wherein generating the digital simulation comprises:
receiving the at least one lighting parameter, the at least one activity parameter, the at least one duration parameter, the at least one personal parameter, and the at least one target vision condition;
generating a parameter rule set based on the parameters received to determine a relationship between a display background and a plurality of images, wherein the display background and the plurality of images are selected from a database;
computing a visual behavior function; and
generating the digital simulation.

15. The computer program product according to claim 14, wherein computing the visual behavior function comprises:
computing a spatial-temporal position of an object, wherein the spatial-temporal position of the object includes at least one rule of distance and at least one rule of angle; and
computing a duration of the object, wherein the duration of the object includes at least one rule of position and at least one effect rule.

16. The computer program product according to claim 13, wherein analyzing the recorded pupillary movement comprises:
measuring a size of the user pupillary opening;
measuring the user pupillary movement and perceived distance and movement of objects generated in the simulation; and
determining the visual comfort assessment, wherein the visual comfort assessment is either positive or negative.

17. The computer program product according to claim 16, wherein the message of the visual comfort assessment comprises either a positive visual comfort assessment and a set of usage guidelines, or a negative visual comfort assessment.

18. The computer program product according to claim 13, wherein the user wears a prescription corrective lens.
